(19) United States
(12) Patent Application Publication
(10) Pub. No.: US 2009/0043150 A1
(43) Pub. Date: Feb. 12, 2009

(54) Method of Preventing and Treating Emotional and Behavioral Problems

(76) Inventor: Laurel Mellin, San Rafael, CA (US)

Correspondence Address: STEVEN A. NIELSEN;ALLMAN & NIELSEN, P.C, 100 Larkspur Landing Circle, Suite 212, LARKSPUR, CA, 94939, US

(21) Appl. No.: 12/189,075

(22) Filed: Aug. 8, 2008

Related U.S. Application Data
(60) Provisional application No. 60/955,313, filed on Aug. 10, 2007.

(51) Int. Cl. A61M 21/02

(52) U.S. Cl. 600/26

(57) ABSTRACT

The method enables individuals to monitor their state based on a five-point stress-reward scale, use the intrapsychic tool that corresponds with that state and continue that process of monitoring, identifying and using until they attain the state of lowest stress and greatest reward or until they accept the other state they are in. That acceptance can trigger them to attain the state of lowest stress and greatest reward. They do this 10 or more times per day. The process triggers immediate improvements in emotional and behavioral regulation that are short lived as this regulation is affected by neural circuitry. The intensive use of the system over time changes the neural circuitry so that attaining the state of lowest stress and greatest reward occurs spontaneously, more frequently and more easily. This brain retraining supports long-term improvements in emotional and behavioral regulation, and ultimately, health and happiness.

### CROSS-REFERENCE TO RELATED APPLICATIONS

This application claims the content, benefit and priority of U.S. provisional patent application No. 60/955,313 filed on Aug. 10, 2007 and entitled, “Method of Preventing and Treating Emotional and Behavioral Problems” the contents of which are incorporated herein by reference.

### STATEMENT REGARDING FEDERALLY SPONSORED RESEARCH OR DEVELOPMENT

Not Applicable

### BACKGROUND OF THE INVENTION

(1) Field of the Invention

Generally, the invention relates to means and methods of preventing and treating a broad range of emotional and behavioral problems that impact health in humans. More particularly, the invention relates to a five or so point system of measuring and regulating states of stress and related emotional and behavioral patterns. The invention relates to ways of preventing or treating emotional problems, such as depression and anxiety, behavioral problems such as overeating and inactivity and health problems, such as obesity and hypertension for which a patient's emotional state and behavioral practices are contributing or exacerbating factors.

(2) Description of the Related Art

Many attempts to monitor regulate, or change human behavior are known in the related art. However, the related art fails to provide the advantages of the present invention. Interventions or other methods in the related art include cognitive-behavioral therapy, psychotherapy, meditation, hypnotherapy, environmental modification, medical procedures, drug therapies, 12-step programs and health education. Currently, all methods in the related art show limited long-term post-treatment effectiveness and other shortfalls.

### BRIEF SUMMARY OF THE INVENTION

The invention may be referred to as, and is “The 5-Point System of Emotional and Behavioral Regulation”, “The Solution”, “The Solution Method”, the “method”, or the “present invention”.

The method involves taking specific acts repeatedly and frequently to monitor and modify one's state of stress using a five point system. The system classifies the continuum of stress states from homeostasis to allostasis into five states, with “1” indicating homeostasis and reward and “5” indicating allostasis. The patient chooses to accept their state or change their state. If they choose to change their state, then they use the specific tool that corresponds to their state.

Rather than focusing on their psychopathology, improving their maladaptive behaviors or their health problem directly, they focus on using this method repeatedly to monitor and change their state to decrease the frequency and duration and allostasis and increase the frequency and duration of homeostasis. Taking this action by using the method results in improvement in emotional, behavioral and health problems.

The method involves specific tools and specific ways of using those tools. The ways of using the tools is based on neuroplasticity, using the tools with the frequency, duration and intensity required for neural retraining. The tools reflect emerging the emerging scientific literatures on neuroscience (cognitive and emotional brain processing) and attachment theory.

Attachment theory has shown that a parent's repeated attunement to the inner state of the child during episodes of the whole range of states and effectiveness in regulating those states is associated with positive emotional, behavioral and health outcomes. Effective regulation increases the frequency and duration of homeostasis. Homeostasis signifies the state of optimal or adaptive emotional, behavioral and physiological functioning and is the object or goal of self-regulation. It decreases the frequency ad duration of allostasis.

Allostasis signifies the body's effort to maintain stability during stress and signifies the state of sub-optimal or maladaptive emotional, behavioral and physiological functioning. Allostasis is adaptive when experienced in response to acute threats (“acute stress”) to survival, but is maladaptive when it represents a chronic state (“chronic stress”) and/or an amplification of stress that is inconsistent with the acute threat to survival.

I drew upon attachment theory in the development of these tools as the scientific literature linking secure attachment/parental attunement and improved health outcomes is substantial. Also, developing tools that approximate parent-child attunement is consistent with the evolution. The capacity of the parent to effectively and consistently regulate and modify the state of the child is associated with survival and it would make sense that natural selection would favor a process that was most effective in promoting resiliency and homeostatic states.

Effective parental regulation of state in childhood is predictive of effective self-regulation of state in adulthood. Emotional self-regulation is related to behavioral regulation and both are related to health status

The patient's use of the tools replicate parental attunement. The skills are used frequently to monitor the patient's state much like the frequent monitoring of a child's state by an attuned parent. The five tools were developed based on emerging understanding of cognitive and emotion processing of input by the brain in each of the five states to effect an efficient and prompt return of the patient to homeostasis.

An object or goal of the invention is to be in the state of homeostasis and reward (“1”) as much of the time as possible by: 1) strengthening the neural circuits that favor that state and weakening those that favor the four other states, 2) strengthening the neural networks that favor resilience (a rapid recovery from stress states), and 3) strengthening the neural networks that favor adaptive expectations and weakening those that favor maladaptive expectations. This is accomplished by using the disclosed 5-point system, preferably at least 10 times daily.

The process of using The 5-Point System involves: 1) monitoring ones state; 2) determining which of five states one is in; 3) determining if they will accept their state or change their state; 4) if determining the latter, using the tool that corresponds to that state; 5) monitoring ones state again; and 6) repeatedly identifying the state and using the tools until in State #1 or in acceptance of State #2-#5. If their choice at any point in the series of actions they take is to accept their state, that acceptance an improvement in state.

Each episode of the system or system use triggers beneficial effects to stress and related emotional and behavioral regulation. Each episode weakens the neural networks that favor states #2 to #5 and strengthens the neural networks of State #1. With the long-term use of the system, the dominant neural networks may favor State #1 and the frequency and duration of states #2 to #5 may decrease. By repeatedly changing their state, they may change their trait. The process may become integrated into the neural circuitry and unconsciously used with conscious use of the tools occurring less frequently than when use of the method began. The goal is to promote persistent improvements in post-treatment emotional and behavioral regulation, decreased symptoms of chronic stress including emotional, behavioral and physical problems.

The present invention overcomes shortfalls in the related art by offering a treatment that retrains the brain to stay in a state that impacts a wide range of emotional, behavioral, relational and health problems. Current treatments tend to be symptom specific with discouraging post-treatment outcomes in that symptom or improved outcomes with symptom substitution (e.g., the smoker becomes the overeater). The proper use of the present invention results in tangible physical changes in patients including but not limited to, decreases in obesity, decreases in blood pressure, increases in physical fitness and decreases in depression.

Current treatments in the related art are dependent upon ongoing therapy and different from the present invention and the disclosed method or system which is educational, training the individual to use the skills to train his or her own brain.

Some current treatments for emotional and behavioral regulation involve devices, drugs, and/or surgery. The disclosed method involves no devices, drugs or surgery, is low cost, non-invasive and minimizes the use of professional intervention. Self-study means of learning the system, such as workbooks, audio programming, books, Internet training, interactive Internet communities and self-help groups may be used. Group programs and individual coaching on this system is possible for those who have the resources to pay for them or whose behavioral or emotional symptoms post a significant medical risk.

These and other objects and advantages will be made apparent when considering the following detailed specification when taken in conjunction with the drawings.

### BRIEF DESCRIPTION OF THE DRAWINGS

FIG. 1 is a flow chart of a 5-point system consistent with the principles of the invention.

FIG. 2 is a summary of the disclosed tools or methods consistent with the principles of the invention.

FIG. 3A and 3B comprise a summary form of one embodiment of the invention suitable for flash card or portable form.

### DETAILED DESCRIPTION OF THE INVENTION

In the following description, for purposes of explanation, numerous specific details are set forth in order to provide a thorough understanding of the invention. It will be apparent, however, to one skilled in the art that the invention can be practiced without these specific details. In other instances, structures and devices are shown in block diagram form in order to avoid obscuring the invention.

The reference in the specification to “one embodiment” or “an embodiment” means that a particular feature, structure, or characteristic described in connection with the embodiment is included in at least one embodiment of the invention. The appearances of the phrase “in one embodiment” in various places in the specification are not necessarily all referring to the same embodiment nor are separate alternative embodiments mutually exclusive of other embodiments.

In the following detailed description of embodiments of the invention, reference is made to the accompanying drawings in which like references indicate similar elements, and in which is shown by way of illustration specific embodiments in which the invention may be practiced. These embodiments are described in sufficient detail to enable those skilled in the art to practice the invention, and it is to be understood that other embodiments may be utilized and that logical, mechanical, electrical, functional, and other changes may be made without departing from the scope of the present invention. The flowing detailed description is, therefore, not to be taken in a limiting sense, and the scope of the present invention is defined only by the appended claims.

The description, which follows, and the embodiments described therein, are provided by way of illustration of an example, or examples of particular embodiments of the principles of the present invention. These examples are provided for the purposes of explanation, and not of limitation, of those principles of the invention. In the description, which follows, like parts are marked throughout the specification and the drawings with the same respective reference numerals. The drawings are not necessarily to scale and in some instances proportions may have been exaggerated in order to more clearly depict certain features of the invention.

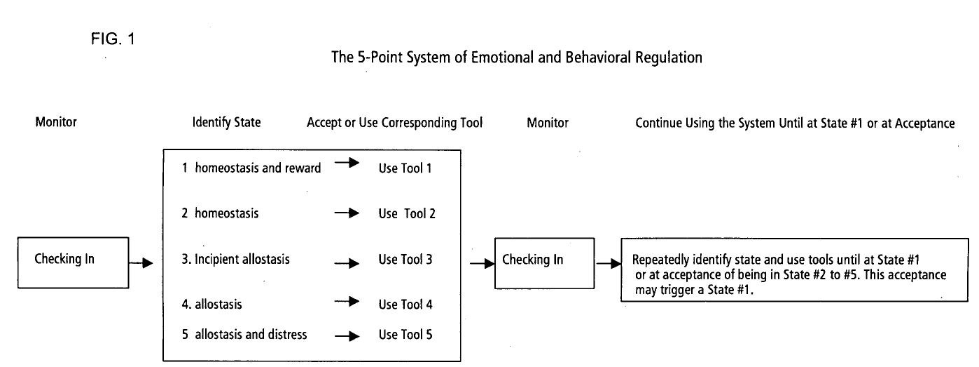

Referring to FIG. 1, individuals monitor their state and determine which of five states they are in, and use the tool that corresponds to that state. Then they monitor their state again. If they are at “State #1”, no further use of the system is needed during that episode. If they are at “State #2” to “State #5”, they repeat the same process, using the corresponding tool until they are at “1” or they feel acceptance of the state they are in. That feeling of acceptance triggers them to State #1. They do this at least 10 times per day until it becomes automatic to monitor and stay in State #1 most of the time.

This method, developmental skills training (DST), has been applied for childhood obesity as The Shapedown Program® and for adult stress-related emotional and behavioral problems as The Solution® and The Solution Method™. I developed the method and authored all the workbooks, audio programming, website designs, professional training, videotapes and other educational material based on the method.

Previous to this application, a prior method of the inventor was comprised of nurturing skills (How do I feel? What do I need? Do I need support), limit-setting skills (Are my expectations reasonable? Is my thinking positive and powerful? What is the consequence of following through? What is the reward?) and related skills including skills for lifestyle change, effective health care and interpersonal training.

The aim of the prior method was for participants to be “in balance” more of the time as the state of being “out of balance” promoted emotional and behavioral dysregulation. The work called that being “above the line” or “below the line.” When individuals were above the line, they would use the nurturing skills and when they were below the line, they would use a cycle, a skill that is the combination of nurturing and limit-setting. Recently, I added a skill involving expressing the eight basic positive and negative feelings to be used when they were neither clearly in balance or out of balance. They would use these skills with an unspecified frequency.

This intervention was beneficial, however, given the science on both neuroplasticity and attachment theory, it was clear to me that a new method would be needed that would provide for intensive, frequent monitoring to change ones state, a system of identifying stress level and tools that corresponded to each stress level that reflected secure attachment.

However, even this method that was not reflective of the emerging science and did not offer the above characteristics has shown remarkable effectiveness. However, the problems that the method targets are considered intractable and refractory. For example, even using comprehensive, expensive, and high-risk interventions for obesity, weight during post-treatment typically rebounds. Although the former method has shown improved outcomes compared to outcomes associated with traditional therapies, I grew discontented with the effectiveness of the method, because it was not based on the emerging science and because those who were attracted to the method tended to be very high risk emotionally, behaviorally and medically. It seemed very important to develop a new method that would be consistent with an integration of these two literatures.

I became aware of emerging research that showed that behavioral dysregulation was often addictive, that the reward circuitry in the brain was stimulated by various excesses and pleasure pathways became less responsive to natural sources of pleasure.

The brain is reward driven and without a substitute pleasure from natural sources, cravings and appetites for common behavioral excesses would be likely to continue. In my clinical evaluation of the programs participants, I concluded that many were not using the skills with the rigor and vigor needed and that most were, at best, attaining “balance” but not high levels of natural rewards. Also, my evaluation showed that when many participants were in the stress response, they were often in a full blown response and unable to use the nurturing and limits cycle. That resulted in a longer duration of the stress response.

In addition, I became aware of emerging research in neurophysiology that supported neuroplasticity and neurogenesis, the changing of the brain with strengthening and weakening of existing neural networks and creating new ones. Changing the brain took specificity, repeating the same experiences, particularly when they were emotionally salient and volitional. Without that highly specific and repetitive training, change would be slow to occur and the beneficial outcomes would be more modest. A more intensive and directed method was needed as in response to stimulation, neural networks compete. In response to stimulation, neural networks compete and the dominant ones fire. The more often they fire, the stronger the wiring becomes and the weaker the competing neural networks become. The more often the dominant ones fire, the stronger the wiring becomes and the weaker the competing neural networks become, stimulating neurogenesis and the strengthening of existing neural networks. An absence of this stimulation weakens existing neural networks.

Last, as I tested this 5-point system, I became aware that participants asked the questions in a depriving or neglectful way. Nurturing and limits skills are meant to enable an individual to mirror a responsive or authoritative parenting style, which includes loving observation. What's more, the emerging scientific literature on the neural networks of an insecure attachment having an important impact on stress made me believe that replicating and practicing a secure, loving attachment as one asks these questions would have efficacy. I developed another tool to systemize a process that mirrored the secure attachment to self, relaxing and lovingly observing themselves, prior to asking the questions of the system. When used repeatedly this would be expected to retrain the brain to create a more secure connection to self.

From these understandings I conceptualized a new system that would provide more structure and direction to give participants a way to effectively retrain their brain for emotional and behavioral regulation that involved:

1. Dividing the Allostasis—Homeostasis Continuum into Five States:

| STATE | SCIENTIFIC TERM | EDUCATIONAL TERM |
|---|---|---|
| State #1 | homeostasis and reward | very above the line |
| State #2 | homeostasis | above the line |
| State #3 | incipient allostasis | on the line |
| State #4 | allostasis | below the line |
| State #5 | allostasis and distress | very below the line |

State #1 involves acute awareness of feelings, thoughts and sensations and the experience of high levels of natural rewards. Drives for common excesses are absent. Mood regulation is high.

State #2 involves some awareness of feelings, thoughts and sensation and a feeling of balance, but not high levels of natural rewards. Drives for common excesses are low. Mood regulation is moderate.

State #3 involves higher levels of stress and awareness of feelings, thoughts and sensations are compromised. Drives for common excesses begin to ramp up and mood regulation begin to suffer.

State #4 occurs when the brain perceives stress and triggers a stress response. Dysregulation of emotions and behavior follows. Drives for common excesses are high.

State #5 occurs when there is a full blow stress response, a flushing of the brain in which there are severe dysregulation of emotions and behavior and feelings of being lost and disoriented. Drives for common excesses feel unstoppable.

2. Identifying a Tool for Each State.

| STATE | TOOL | DESCRIPTION |
|---|---|---|
| State #1 | The Sanctuary Tool | Strengthening Secure |
| | | Positive Connections |
| State #2 | The Feelings Check | Identifying Feelings and Needs |
| State #3 | Emotional Housecleaning | Expressing 8 Basic Feelings |
| State #4 | A Cycle | Expressing 4 Basic Feelings |
| | Constructing Adaptive Limit | |
| | Repetition of New | |
| | Adaptive Limit | |
| State #5 | The Damage Control | Calming emotional crisis |
| | Tool | Re-establishing Secure |
| | | Connections |

Tool Details

Tool #1 The Sanctuary Tool—requires participants to feel high levels of natural reward from feelings, thoughts, sensations, to be present in the moment, feeling securely connected to self and others.

Take deep breaths.

Assume “body above the line” (posture, movements and facial expression of balanced and reward).

Observe yourself, in a loving manner.

Aware of a safe place inside body.

Bring up a nurturing inner voice.

Say to yourself the words you need to hear.

Send love to one other person.

Collect a Joy Point (a spike of neurotransmitters of pleasure)

State #2 The Feelings Check—enables participants to check their feelings, the immediate, accurate information that indicates their needs, to identify the corresponding need and to ask for support to meet that need. To be sued in the state of 2.

Comprises the steps of asking:

How do I feel?

What do I need?

What do I really need?

State #3 Emotional Housecleaning—as stress mounts, the capacity to be aware of feelings diminishes or the feelings become unbalanced. This tool increases awareness of feelings and has a balancing impact on them. Participants express the following feelings: anger, sadness, fear, guilt, gratitude, happiness, security and pride.

Emotional Housecleaning comprises the steps of expressing one or more of each of eight core feelings, wherein a feeling is associated with an event, object, or other person, which may be defined as “x”. For example, Emotional Housecleaning may include making the following statements:

I feel angry that x

I feel sad that x.

I feel afraid that x.

I feel guilty that I x.

I feel grateful that x.

I feel secure that I.

I feel proud that I x.

State #4 A Cycle—during a stress response, a stronger tool is needed and this skill replicates the process that is internalized with responsive parenting. Participants state what they are upset about, express their anger, sadness, fear and guilt. Awareness of those feelings increases access to the implicit memory, the unconscious expectations that are imbedded in the neuronal circuitry from repeated and early experience. The participant identifies the unreasonable expectation that is amplifying the stress, replace it with a reasonable expectation, support that expectation with positive, powerful thoughts, then identify the developmental lesson, the essential pain of following through with that new expectation and identify the reward of doing so. This creates a relief of pain and a surge of pleasure and some learning—the retraining of the basic expectations of life. It has a maturing effect when used over time, hence the term developmental skills training. The skill continues with checking one's needs and asking for support in order to follow through with the new reasonable expectation. The tool concludes with checking if one wants to strengthen the neural networks of the new expectation, that is, to “grind in” the expectation by repeating it 10 times, one to three times daily, for one week, then re-evaluating the need to repeat the process for another week.

The Cycle Tool May Comprise the Steps of:

Stating or writing the thoughts about or the description of what one is distressed about, a Thinking Journal.

The Natural Flow of Feelings: The statements of the first four feelings of the Emotional Housecleaning tool.

Asking:

What is my unreasonable expectation?

What is my reasonable expectation?

Is my thinking positive and powerful?

What is the essential pain?

What is the earned reward?

What do I need?

Do I need support?

Asking:

What is my Grind-in?

The Grind-In statement is the reasonable expectation that replaces the unreasonable one that contributes to stress. Repeating that Grind-In statement 10 times, one to three times daily for one week. Then re-evaluating the need to repeat the process for another week.

State #5 The Damage Control Tool—when there is a flooding of the brain with a stress response, feelings, thoughts and sensations are affected. In addition, since this is a survival response, connections change. Often participants feel lost, panicked, disconnected. This simple tool—repeating: Do not judge. Minimize harm. Know it will pass—enables participants to stop the spiraling stress that is so destructive to emotional and behavioral regulation. Recommended to repeat it 10 times, then check in and assess state.

The Damage Control Tool Comprises the Steps of:

Stating:
Do Not Judge.
Minimize Harm.
Know It Will Pass.

3. Increasing Rigor

The use of the system must be rigorous. Instead of using tools to no specific end other than “getting above the line” which for many participants meant out of acute pain without accessing an abundance of emotional rewards, the new directive is to “get to 1” Don't stop checking in, identifying your state and using the corresponding tool until you are at “State #1” OR you feel accepting of the state you are in. That acceptance often triggers participants to be at “State #1.”

4. Increasing Vigor

The use of the system must be frequent to disrupt the automatic repeated arousal of the dominant neural networks that favor emotional and behavioral dysregulation. A minimum of 10 uses of the system per day is recommended.

5. Improving Quality of Observation

By initiating a use of the skills by taking several deep breaths to relax and better access feelings and consciously observing oneself in a loving way the neural networks that favor the quality of the relationship with self ma begin to change. Instead of a depriving, neglectful or indulging relationship with self, an insecure or maladaptive connection, a more secure connection is supported.

These scientific understandings in addictive behavior, attachment theory and neurophysiology became apparent to me at the same time that I became more aware that the old method was not adequate to offer optimal treatment for high-risk emotional and behavioral problems.

In 2007, 1 conceptualized and began experimenting with this new system. I was astonished by the findings and responses. Participants who had struggled with weight for decades and had tried everything, including the old system of the method, experienced remarkable results. Those who had never been at “State #1” were using the system and attaining it. People who had had years of psychotherapy without any persistent improvement in mood reported mood elevation. The degree to which the results improved and the enthusiasm of our test population were unexpected.

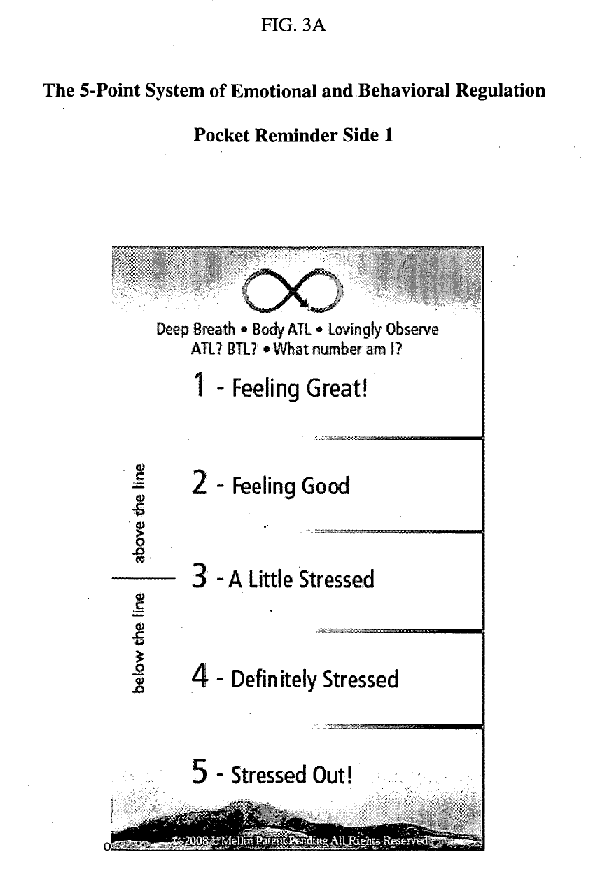

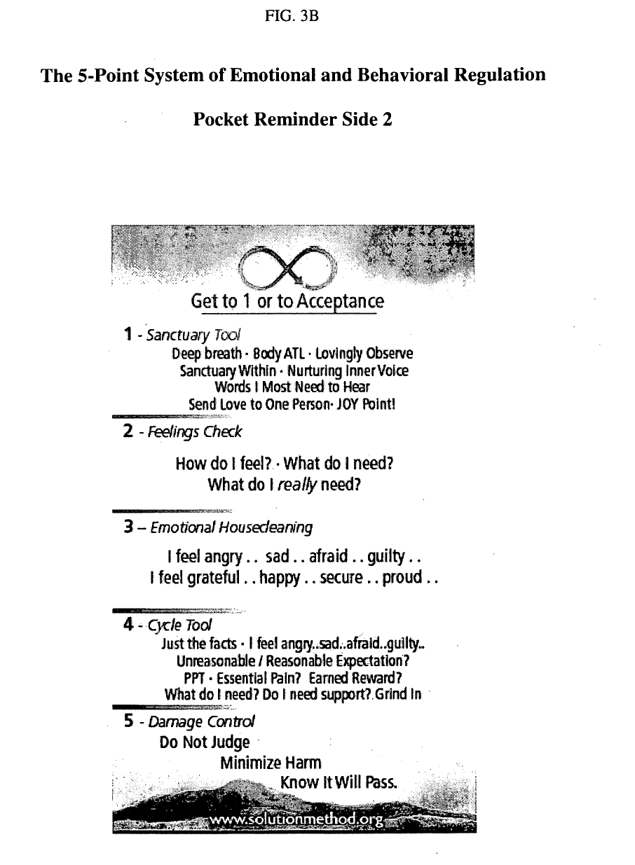

FIGS. 3A and 3B summarize a pocket reminder method which may comprise two sheets of paper or two sides of paper that contain an outline of one embodiment or summary of the invention. Referring to FIG. 3A, emotional states 1 and 2 are above the line and emotional states 4 and 5 are below the line. ATL stands for “above the line” and BTL stands for “below the line. The five emotional states may be summarized as:

1. Feeling Great

2. Feeling Good

3. A Little Stressed

4. Definitely Stressed

5. Stressed Out !

The contents of FIG. 3A may be referred to as “Pocket Reminder Side 1”

Referring to FIG. 3B, which may be referred to as “Pocket Reminder Side 2”, each emotional state of FIG. 3A is linked to a tool described or summarized in FIG. 3B. The following mapping of emotional states to tools and tool summaries may be used:

1. SanctuaryTool
Deep breath, Body ATL, Lovingly Observe Sanctuary Within, Nurturing Inner Voice, Words I Most Need to Hear, Send Love to One Person, Joy Point!

2. Feelings Check
How do I feel? What do I need? What do I really need?

3. Emotional Housecleaning
I feel angry . . . sad afraid . . . guilty.
I feel grateful . . . happy . . . secure . . . proud.

4. Cycle Tool
Just the facts, I feel angry . . . sad . . . afraid . . . guilty.
Unreasonable/Reasonable Expectation?
PPT, Essential Pain? Earned Reward?
What do I need? Do I need support

5. Damage Control
Do Not Judge
Minimize Harm
Know It Will Pass

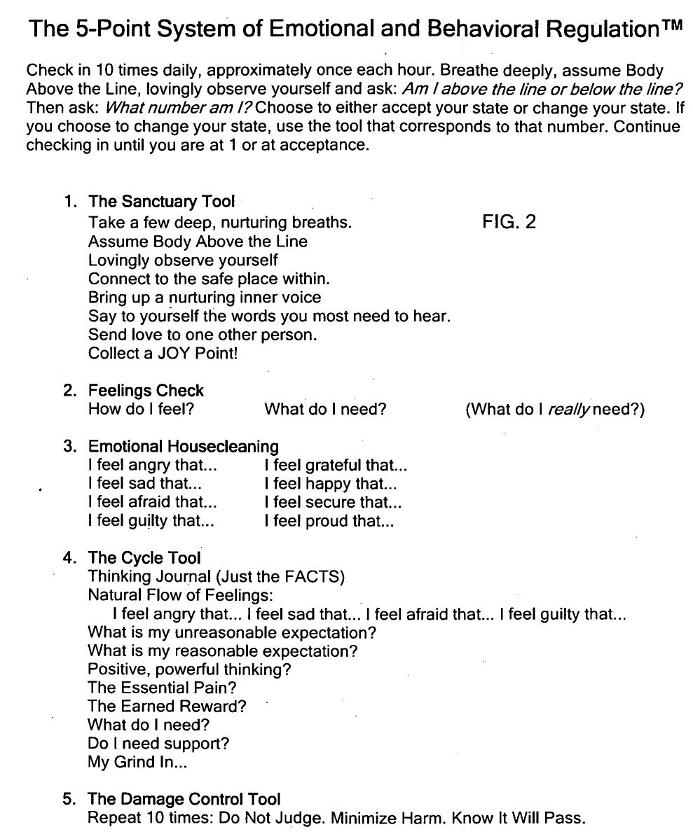

FIG. 2 is an alternative summary of one embodiment of the invention. Each of the five tools is summarized and instructions are given to check one's emotional state ten times a day. The principles of one embodiment of my invention may be summarized as follows:

1. A method of changing behavior comprising the steps of:
a) evaluating state by taking deep breathings and lovingly observing self;
b) assessing current state as in allostasis or homeostasis;
c) assigning an integer value between 1 and 5, to correspond to the present state, wherein experiencing high intensity homeostasis or joy is 1, high intensity stress or pain is 5 and 2,3, and 4 fall in between 1 and 5;
d) using the tool that corresponds to the current state with the following mapping:

I. The Sanctuary Tool

II. Feeling Check

III. Emotional Housecleaning

IV. A Cycle

V. The Damage Control Tool

wherein each tool corresponds to certain procedures as follows:

i. The Sanctuary Tool
a) take 3 or more deep breaths;
b) assume body above the line;
c) lovingly observe yourself;
d) e aware of the safe place inside your body
e) summon or bring up a inner nurturing voice and say to yourself the words that you most need to hear right now;
f) send love to one person;
g) collect a Joy Point.

ii. Feelings Check
a) ask yourself:
   i. how do I feel?;
   ii. what do I need?;
   iii. what do I really need?

iii. Emotional Housecleaning
a) complete the questions:
   i. I feel angry that . . .
   ii. I feel sad that . . .
   iii. I feel afraid that . . .
   iv. I feel guilty that . . .
   v. I feel grateful that . . .
   vi. I feel happy that . . .
   vii. I feel secure that . . .
   viii. I feel proud that . . .

4. A Cycle

a) thinking journal;
b) completing the first four questions of Emotional Housecleaning;
c) asking, what is my reasonable expectation?
d) asking, what is my unreasonable expectation?
e) asking, is my thinking positive and powerful?
f) asking, what is the essential pain?
g) asking, what is the earned reward?
h) asking, what do I need?
i) asking, do I need support?
j) asking, what is my Grind In?

5. The Damage Control Tool

a) state repeatedly:
Do not judge
Minimize harm;
Know it will pass.

What is claimed is:

1. A method of changing behavior comprising the steps of:
a) evaluating state by taking deep breathings and lovingly observing self;
b) assessing current state as in allostasis or homeostasis;
c) assigning an integer value between 1 and 5, to correspond to the present state, wherein experiencing high intensity homeostasis or joy is 1, high intensity stress or pain is 5 and 2,3, and 4 fall in between 1 and 5;
d) using the tool that corresponds to the current state with the following mapping:
I. the sanctuary tool
II. feeling check
III. emotional housecleaning
IV. the cycle tool
V. the damage control tool
wherein each tool corresponds to certain procedures as follows:
i. the sanctuary tool:
a) take three or more deep breaths;
b) assume body above a line;
c) lovingly observe yourself;
d) be aware of the sanctuary within;
e) summon or bring up a inner nurturing voice and say to yourself the words that you most need to hear right now;
f) send love to one other person;
g) collect a Joy Point (intentional surge of neurotransmitters)
ii. feelings check
a) ask yourself:
how do I feel?;
what do I need?;
what do I really need?
iii. emotional housecleaning
a) complete the questions:
i. I feel angry that . . .
ii. I feel sad that . . .
iii. I feel afraid that . . .
iv. I feel guilty that . . .
v. I feel grateful that . . .
vi. I feel happy that . . .
vii. I feel secure that . . .
viii. I feel proud that . . .
4. the cycle tool:
a) performing a thinking journal, wherein one states or writes their thoughts or a description of what one is distressed about;
b) completing the first four questions of emotional housecleaning;
c) asking, what is my reasonable expectation?
d) asking, what is my unreasonable expectation?
e) asking, is my thinking positive and powerful?
f) asking, what is the essential pain?
g) asking, what is the earned reward?
h) asking, what do I need?
i) asking, do I need support?
5. damage control tool:
a) state:
do not judge;
minimize harm; and
know it will pass.

2. The method of claim 1 used for losing weight or decreasing depression.

3. A method of treating behavioral problems, such as obesity and depression; the method comprising:
a) monitoring ones state;
b) determining which of five states one is in, wherein the five states are defined as:
1. homeostasis and reward;
2. homeostasis;
3. incipient allostasis;
4. allostassis;
5. allostasis and distress;
c) mapping or identifying a tool for each state as follows:
1) state 1 (homeostasis and reward) maps to sanctuary tool;
2) state 2 (homeostasis) maps to feeling check tool;
3) state 3 (incipient allostasis) maps to emotional housecleaning tool;
4) state 4 (allostassis) maps to cycle tool;
5) state 5 (allostasis and distress) maps to damage control tool;
d) performing the act or actions mapped to each tool as follows:
1) sanctuary tool: take three or more deep breaths;
2) feeling check tool: ask yourself, how do I feel ?;
3) emotional housecleaning tool: complete the following statement, I feel angry that . . . ;
4) cycle tool: complete the emotional housecleaning tool and then create a grind-in statement that is a reasonable expectation to replace an unreasonable expectation that is causing stress, then repeat the grind in statement one or more times;
5) damage control tool: say out loud, “do not judge”.

4. The method of claim 3 wherein the damage control tool prompts the user to state “do not judge, minimize harm, and know it will pass.”

5. The method of claim 4 used until one is in state 1(homeostasis and reward).

6. A method of treating behavioral problems, such as obesity and depression; the method comprising:
a) checking one's emotional state 10 or more times per day;
b) selecting one description of an emotional state, from the group comprising:
i. feeling great;
ii. feeling good;
iii. a little stressed;
iv. definitely stressed;
v. stressed out;
c) performing one or more actions mapped to the emotional state selected, wherein each emotional state is assigned an action comprising:
i. take a deep breath;
ii. ask “what do I really need”;
iii. complete the statement “I feel angry because . . . ”;
iv. ask “do I need support”;or
v. say “do not judge”.
